(19) United States
(12) Patent Application Publication
(10) Pub. No.: US 2009/0317327 A1
(43) Pub. Date: Dec. 24, 2009

(54) Aqueous Dispersion of Superparamagnetic Single-Domain Particles, Production and Use Thereof in Diagnosis and Therapy

(75) Inventor: Herbert Pilgrimm, Berlin (DE)

Correspondence Address: NORRIS, MCLAUGHLIN & MARCUS, P.A., 875 THIRD AVE, 18TH FLOOR, NEW YORK, NY, 10022, US

(73) Assignee: FERROPHARM GMBH, TELTOW (DE)

(21) Appl. No.: 12/096,692

(22) PCT Filed: Dec. 7, 2006
(86) PCT No.: PCT/EP06/69453

§ 371 Date: Jan. 29, 2009

(30) Foreign Application Priority Data
Dec. 9, 2005 (DE) 10 2005 059 751.3

(51) Int. Cl. A61K 51/00; A61K 47/02

(52) U.S. Cl. 424/1.89; 514/769; 424/1.81; 424/1.85

(57) ABSTRACT

The invention relates to an aqueous dispersion of superparamagnetic iron-containing particles bearing α-hydroxycarboxylic acids as stabilizer substances on their surface, said dispersion comprising N-methyl-D-glucamine (meglumine) and/or 2-amino-2-(hydroxymethyl)-1,3-propanediol (trometamol) and the content of free iron ions being lower than 1 mg of iron per liter. In a preferred embodiment the dispersion according to the invention may additionally include an iron-complexing agent. In another preferred embodiment the dispersion includes positively charged metal ions and/or compounds containing polyamino groups, which can be bound to substances having a therapeutic or diagnostic effect. The invention is also directed to a method of producing said dispersion, the use thereof as an MRT contrast medium as well as the use thereof as therapeutic agent, including the option of therapy follow-up using an imaging method.

The invention relates to an aqueous dispersion of superparamagnetic iron-containing particles bearing α-hydroxycarboxylic acids as stabilizer substances on their surface, said dispersion comprising N-methyl-D-glucamine (meglumine) and/or 2-amino-2-(hydroxymethyl)-1,3-propanediol (trometamol) and the content of free iron ions being lower than 1 mg per liter iron. In a preferred embodiment the dispersion according to the invention may additionally include an iron-complexing agent. In another preferred embodiment the dispersion includes positively charged metal ions and/or compounds containing polyamino groups, which can be bound to substances having a therapeutic or diagnostic effect.

The invention is also directed to a method of producing said dispersion, the use thereof as a MRT contrast medium as well as the use thereof as therapeutic agent, including the option of therapy follow-up using an imaging method.

In recent years, “molecular imaging”, i.e. in vivo characterization and representation of biological processes on a cellular and molecular level, has gained more and more importance in the investigation of diseases and increasingly in clinical application as well. The basis of this is the development of molecular markers capable of detecting the desired molecular targets in a sufficiently sensitive manner, using imaging techniques that are available or to be developed.

Owing to its excellent soft-tissue contrast compared to other imaging techniques, and having high anatomic resolution at the same time, magnetic resonance tomography (MRT) has been established as an important pillar of clinical-radiological diagnostics. With the introduction of superparamagnetic iron oxide nanoparticles having high T2 and some T1 relaxivity, efficient markers for molecular imaging have become available.

The patent applications WO-A-96/03653, WO-A-97/35200 and WO-A-2004/034411 describe very small superparamagnetic iron oxide particles, referred to as VSOPs (very small iron oxide particles), which are well suited for molecular imaging and drug targeting.

VSOPs are significantly smaller compared to the previously known polymer-coated (using e.g. dextran) superparamagnetic iron oxide particles (SPIO, USPIO). For example, citrate-coated VSOPs have a hydrodynamic diameter of ˜7 nm, while the smallest polymer-coated USPIOs have a diameter of about 15 to 20 nm.

However, these previously known small superparamagnetic iron particles neither have optimum tolerability in an animal or human body when administered on the parenteral or enteric route.

Trivalent and, in particular, bivalent iron ions are highly toxic to biological tissue and to mammals and humans. Thus, the toxicity of manganese-iron ferrites stabilized with citric acid was found to be very high (Lacava et al., Biological effects of magnetic fluids: toxicity studies, J of Magnetism a. Magnetic Materials, 201 (1999) 431-434).

This is also familiar from the use of iron complexes in parenteral iron replacement therapy in iron deficiency anemia. Thus, intravenous injection of an iron-sucrose complex as active substance in the finished, approved drug Venofer® results in temporary renal damage triggered by oxidative stress caused by free iron ions (Agarwal et al., Kidney International, 2004 Vol. 65: 2279-2289). Furthermore, free iron ions have a toxic effect on red blood cells (risk of hemolysis).

Apart from the direct cell-damaging effect of free iron ions, well-known clinically approved preparations for iron replacement therapy as well as clinically approved iron oxide particles for MR diagnostics exhibit the side-effect spectrum of anaphylactic reaction induced by polymer stabilizer substances such as dextran.

While Endorem® (AMI 227) from Laboratoire Guerbet (France), which cannot be stabilized by heat, has been developed on the basis of superparamagnetic iron oxide nanoparticles, it has been stabilized with dextran and is therefore highly intolerable. Due to the intolerability, it may only be used by infusion with a glucose solution and at a low concentration of 20 μmol Fe/kg. It has been approved for the detection of liver tumors using MRT.

Another approved liver-specific superparamagnetic iron oxide particle is Resovist® from Schering AG (Germany). It involves relatively large dextran-coated superparamagnetic iron oxide particles which, following application, are immediately absorbed by the macrophages of the liver. Consequently, these particles circulate in the blood only for a very short time. Intolerance may occur despite the low dosage of 20 μmol Fe/kg.

The object of the invention was therefore to provide an aqueous dispersion of very small superparamagnetic iron-containing particles, which provides a high contrast effect but is less toxic so that even parenteral use is possible without side effects. Also, the dispersion should be heat-sterilizable without substantially increasing the concentration of free iron ions and without loss of effectiveness of the iron-containing particles and deterioration of the contrast. In addition, the iron-containing particles should have a prolonged residence time in the blood.

The object of the invention is accomplished in accordance with the claims. The subclaims represent preferred embodiments of the respective independent claims. Accordingly, an aqueous dispersion of single-domain particles of iron hydroxide, iron oxide hydrate, iron oxide, iron mixed oxide or iron with a particle size of from 2 to 10 nm is provided, which particles bear aliphatic di- and/or tricarboxylic acids selected from citric acid, malic acid, tartaric acid, derivatives or mixtures thereof as stabilizer substances on their surface, which dispersion is characterized in that it comprises N-methyl-D-glucamine (meglumine) and/or 2-amino-2-(hydroxymethyl)-1,3-propanediol (trometamol) and that the content of free iron ions is lower than 1 mg per liter iron. The dispersion can be produced by precipitation of the iron-containing particles from aqueous iron salt solutions using an alkali solution or ammonium hydroxide, subsequent treatment with the above-mentioned di- and/or tricarboxylic acids, derivatives or mixtures thereof and purification of the particles thus stabilized using dialysis with distilled water until the dialyzate has an electric conductivity of less than 10 μS/cm. The dispersion thus obtained will be referred to as prepurified dispersion. Thereafter, inventive treatment of the prepurified dispersion with aqueous solutions of salts of aliphatic di- and/or tricarboxylic acids selected from citric acid, malic acid, tartaric acid, derivatives or mixtures thereof is effected and dialysis with distilled water is performed until the dialyzate has an electric conductivity of less than 10 μS/cm and a content of free iron ions of less than 1 mg/l. Subsequently, the dispersion is treated with an aqueous solution of the above-mentioned free di- and/or tricarboxylic acids, derivatives or mixtures thereof and dialyzed with distilled water until the dialyzate has an electric conductivity of less than 10 μS/cm and a content of free iron ions of less than 1 mg/l, and N-methyl-D-glucamine (meglumine) and/or 2-amino-2-(hydroxymethyl)-1,3-propanediol (trometamol) are added.

Surprisingly, it was found that the toxicity of an aqueous dispersion of superparamagnetic single-domain particles can be strongly reduced by treatment of the prepurified and stabilized particles (as described in WO 97/35200, for example) with solutions of tri-, di- and mono-salts of aliphatic di- and/or tricarboxylic acids and subsequent dialysis with distilled water, followed by treatment with solutions of free di- and/or tricarboxylic acids and subsequent dialysis with distilled water. These measures reduce the concentration of free iron ions in the ultrafiltrate by magnitudes, so that the very small particles according to the invention can be used with advantage for parenteral administration in humans. Further reduction in toxicity is achieved by adding N-methyl-D-glucamine (meglumine) and/or 2-amino-2-(hydroxymethyl)-1,3-propanediol (trometamol).

In a preferred embodiment of the invention, citric acid is used as aliphatic tricarboxylic acid. As citrates, trisodium citrate and disodium hydrogen citrate are preferably used.

The superparamagnetic single-domain particles stabilized e.g. with citric acid form a stable magnetic fluid in aqueous dispersion, which is prepurified from water-soluble reaction products formed during the production of the superparamagnetic single-domain particles, using dialysis against distilled water. This procedure has been described in WO 97/35200, and the particles thus obtained will be referred to as stabilized and prepurified particles in the present description.

In a preferred embodiment of the invention, the prepurified and stabilized superparamagnetic single-domain particles are then treated with aqueous solutions of tri-, di-, or mono-salts of citric acid to reduce the content of free iron ions and subsequently dialyzed with distilled water, then treated with an aqueous solution of free citric acid, and subsequently redialyzed with distilled water until the content of free iron ions is less than 0.005% of the overall amount of iron.

Surprisingly, it was also found that the residence time of the superparamagnetic iron-containing particles according to the invention in the blood is prolonged by such treatment with solutions of salts of di- or tricarboxylic acids and of the free acids, followed by dialysis with distilled water each time.

According to the invention, compounds containing monoamino groups, selected from D-(−)-N-methylglucamine (meglumine) or 2-amino-2-(hydroxymethyl)-1,3-propanediol (trometamol) or a mixture thereof, are bound to the iron-containing particles stabilized in this way. Surprisingly, it was found that complete or partial replacement of the cations, such as ammonium, sodium or hydronium ions of the free carboxyl groups in the particles stabilized with e.g. citric acid, with N-methyl-D-glucamine and/or 2-amino-2-(hydroxymethyl)-1,3-propanediol results in reduced toxicity of the iron-containing particles according to the invention.

In another embodiment of the invention the dispersion can be added with physiologically tolerable compounds containing polyamino groups, selected from the group comprising polyethyleneimines (PEI), polyvinylamines (PVAm), PEI and PVAm copolymers, polylysine, spermine, spermidine, protamin, protamin sulfate, oligopeptides, polypeptides, denaturation products of proteins and proteids such as gelatin, casein hydrolyzates, glutelins; nitrogen-containing polysaccharides such as mucopolysaccharides, glycoproteids, chitins and mixtures thereof, preferably polyethyleneimines (PEI) or polyvinylamines (PVAm).

As a result of partial replacement of the cations, such as ammonium, sodium or hydronium ions of the free carboxyl groups in the iron-containing particles stabilized with e.g. citric acid, with compounds containing polyamino groups, diagnostically effective substances, cell- and tissue-specific binding substances, pharmacologically active substances, pharmacologically active cells or cell fusion-mediating substances can be chemically bound to said compounds containing polyamino groups according to well-known coupling methods. Initially, the biologically active substances can be bound to the polyamines and purified, and the reaction products can subsequently be coupled to the iron-containing particles of the invention.

As diagnostically effective substances, e.g. fluorescent dyes for a wavelength range of from 200 to 1,200 nm can be bound to the polyamines to combine MRT with optical diagnostic methods. For example, fluorescein, Rhodamine Green, Texas Red as well as mixtures thereof are possible as fluorescent dyes.

Similarly, diagnostically effective substances such as perfluoro molecules used in ultrasonic diagnostics can be bound to the compounds containing polyamino groups. For example, perfluoroalkyl phosphate, perfluoroalkoxypolyethylene glycol phosphate, hexafluorophosphate as well as mixtures thereof are possible as perfluoro molecules.

For example, short-lived radiopharmaceutical agents used to combine MRT with positron emission tomography (PET) can be bound as diagnostically effective substances to the polyamines. Organic substances containing ¹¹C, ¹³N, ¹⁵O, ¹⁸F, ⁶⁸Ga, ⁷⁵Br, ¹²³I, such as [¹¹C]-thymidine, [¹⁸F]-fluoro-L-DOPA, [⁶⁸Ga]-anti-CD66, find use as radiopharmaceutical agents.

As cell- or tissue-specific binding substances, e.g. antigens, antibodies, ribonucleic acids, deoxyribonucleic acids, ribonucleic acid sequences, deoxyribonucleic acid sequences, haptens, avidin, streptavidin, protein A, protein G, annexin, endotoxin-binding proteins, lectins, selectins, integrins, surface proteins of organelles, viruses, microbes, algae, fungi, as well as mixtures thereof, can be bound to the compounds containing polyamino groups.

As pharmacologically active substances, e.g. antitumor proteins, enzymes, anti-tumor enzymes, antibiotics, plant alkaloids, alkylation reagents, anti-metabolites, hormones and hormone antagonists, interleukins, interferons, growth factors, tumor necrosis factors, endotoxins, lymphotoxins, integrins, urokinase, streptokinase, plasminogen-streptokinase activator complex, tissue plasminogen activators, Desmodus plasminogen activators, macrophage activation bodies, antisera, blood and cell constituents and degradation products and derivatives thereof, cell wall components of organelles, viruses, microbes, algae, fungi and degradation products and derivatives thereof, protease inhibitors, alkyl phosphocholines, substances containing radioactive isotopes, surfactants, cardiovascular pharmaceutical agents, chemotherapeutic agents, gastrointestinal pharmaceutical agents, neuropharmaceutical agents, as well as mixtures thereof can be bound to the compounds containing polyamino groups.

As pharmacologically active cells, e.g. organelles, viruses, microbes, algae, fungi, in particular erythrocytes, thrombocytes, granulocytes, monocytes, lymphocytes, and Langerhans islands, can be bound to the compounds containing polyamino groups.

Binding these substances to the compounds containing polyamino groups is well-known to those skilled in the art. Thus, for example, the covalent bond of the compounds containing polyamino groups or their reaction products with the above-mentioned substances with the inventive single-domain particles stabilized by means of e.g. citric acid can be formed using e.g. substances from the group of carbodiimides, such as 1-ethyl-3-(3-dimethylaminopropyl)carbodiimide (EDC), 1-cyclohexyl-3-(2-morpholinoethyl)carbodiimide (CMC) or dicyclohexyl-carbodiimide (DCC). In this way, stable linkages between the carboxyl groups of the stabilizer molecules on the surface of the single-domain particles according to the invention and the amino groups of the above-mentioned substances can be created.

Non-covalent coupling may proceed via electrostatic interactions. For example, polyamines bind electrostatically to citrate-coated iron-containing particles.

In another embodiment of the invention the dispersion according to the invention contains positively charged metal ions of chemical elements such as copper, silver, gold, iron, gallium, thallium, bismuth, palladium, rhenium, ruthenium, platinum, technetium, indium, iridium, radium, selenium, yttrium, zirconium and rare earths, as well as mixtures thereof, and the metal ions can also be radioactive isotopes of said chemical elements, such as ⁵²Fe, ⁶⁷Ga, ^{99m}Tc, ¹¹³In, ¹⁸⁸Rh, ¹⁹²Ir, ¹⁹⁸Au, ²⁰¹Tl, ²²³Ra, as well as mixtures thereof. In addition, the above-mentioned compounds containing mono- and/or polyamino groups, as well as diagnostically effective substances, cell- and tissue-specific binding substances, pharmacologically active substances, pharmacologically active cells or cell fusion-mediating substances can be bound to these particles.

In another embodiment of the invention the toxicity of the inventive aqueous dispersion of iron-containing particles is further reduced by adding a physiologically tolerable iron-complexing agent to the galenic formulation and, surprisingly, there is no dissolution of the particles. Preferred complexing agents are e.g. glycerol-phosphoric acid, ethylenediaminetetraacetic acid (EDTA), N-hydroxyethyl-ethylenediaminetriacetic acid (HEDTA), diethylenetriaminepentaacetic acid (DTPA), α-mercaptopropionylglycine (thiopronine), 2,3-mercapto-1-propane-sulfonic acid, 30-amino-3,14,25-trihydroxy-3,9,14,20,25-pentaazatriacontane-2,10,13,21,24-pentaone-methanesulfonic acid (deferoxamine mesylate). As a result of their low toxicity, these particles/dispersions are also suitable for multiple applications in humans, e.g. in parenteral iron replacement therapy. Accumulation of these particles in organs of the hemopoietic system (bone, marrow, spleen) results in a depot effect, thus providing an advantageous therapy in patients suffering from iron deficiency diseases. The concentration of the physiologically tolerable iron-complexing agent in the dispersion is in the range of from 1 to 20 wt. %, relative to the content of iron.

As cations of the iron-complexing agent containing acid groups, it is possible to use sodium, potassium, calcium, magnesium, D-(−)-N-methylglucamine (meglumine) or 2-amino-2-(hydroxymethyl)-1,3-propanediol (trometamol) as well as mixtures thereof.

In a preferred embodiment, glycerophosphoric acid or a salt thereof, more preferably sodium glycerolphosphate, is used as complexing agent.

If the iron-containing particles of the invention, having positively charged metal ions bound thereto, are also intended to bear an iron-complexing agent, e.g. a glycerolphosphate, it is important that the positively charged metal ions are bound first, i.e., added to the dispersion, and the complexing agent is added only when producing the galenic formulation.

Binding of radioactive metal ions added to the dispersion, e.g. technetium-99m or gallium-67, on the surface of the superparamagnetic single-domain particles of the invention results in a contrast agent allowing to create a new combination of MRT imaging and nuclear-medical imaging. This new imaging method combines the high resolving power of MR tomography with the high sensitivity of nuclear-medical imaging methods such as scintigraphy or SPECT (Single-Photon Emission Computed Tomography).

Binding of dyes or short-lived radioactive markers allows production of MR contrast media for parenteral application, enabling a combination of MRT and optical imaging or a combination of MRT and nuclear-medical imaging such as PET.

As an alternative option, two separately recorded sets of image data from MRT and optical or nuclear-medical imaging can be assembled to form a single image and used to improve the diagnosis of diseases.

The very small superparamagnetic single-domain particles of the invention may consist of the following substances: iron hydroxide, iron oxide hydrate, Fe₂O₃, Fe₃O₄, iron mixed oxides of general formula mMO.nFe₂O₃, wherein M represents the bivalent metal ions Fe, Co, Ni, Mn, Be, Mg, Ca, Ba, Sr, Cu, Zn, Pt or mixtures thereof, mixed oxides of general formula mFe₂O₃.nMe₂O₃, wherein Me represents the trivalent metal ions Al, Cr, Bi, rare earth metals or mixtures thereof or iron, m and n in each of the above formulas being integers of from 1 to 6. Thus, via composition and structure of the single-domain particles, the magnetic susceptibility thereof can be varied within wide limits and the relaxivity ratio R2/R1 can be adjusted to less than 5.

The invention is also directed to a method for the production of an aqueous dispersion of superparamagnetic single-domain particles of iron hydroxide, iron oxide hydrate, iron oxide, iron mixed oxide or iron with a particle size of from 2 to 10 nm, which particles bear aliphatic di- and/or tricarboxylic acids selected from citric acid, malic acid, tartaric acid, derivatives or mixtures thereof as stabilizer substances on their surface, by precipitation of the superparamagnetic iron-containing particles from aqueous iron salt solutions using an alkali solution or ammonium hydroxide, subsequent treatment with aliphatic di- and/or tricarboxylic acids selected from citric acid, malic acid and tartaric acid, derivatives or mixtures thereof, and purification of the particles thus stabilized using dialysis with distilled water until the dialyzate has an electric conductivity of less than 10 μS/cm, which method is characterized in that the dispersion is subsequently treated with an aqueous salt solution of aliphatic di- and/or tricarboxylic acids selected from citric acid, malic acid and tartaric acid, derivatives or mixtures thereof and dialyzed with distilled water until the conductivity of the dialyzate is less than 10 μS/cm and the content of free iron ions is less than 1 mg/l, the dialyzate is subsequently treated with an aqueous solution of free di- and/or tricarboxylic acids selected from citric acid, malic acid, tartaric acid, derivatives or mixtures thereof and re-dialyzed with distilled water until the conductivity of the dialyzate is less than 10 μS/cm and the content of free iron ions is less than 1 mg/l, and N-methyl-D-glucamine (meglumine) and/or 2-amino-2-(hydroxymethyl)-1,3-propanediol (trometamol) are added.

The very small superparamagnetic single-domain particles are initially produced in a well-known manner by precipitation from aqueous iron salt solutions with alkali solution or aqueous ammonia and subsequent treatment with 20 to 50 wt. % stabilizing acid selected from malic acid, tartaric acid, citric acid, mixtures and derivatives thereof, such as monoethers or monoesters thereof, which prevent aggregation and sedimentation under gravity, and subsequently prepurified by dialysis with distilled water until the electric conductivity of the dialyzate is less than 10 μS/cm. As a result of the inventive treatment of the thus-prepurified superparamagnetic iron oxide particles preferably with solutions of tri-, di-, and mono-salts of citric acid and dialysis with distilled water until the content of free iron ions is less than 1 mg/l and subsequent treatment with an aqueous solution of free citric acid and dialysis with distilled water until the content of free iron ions is less than 1 mg/l, the percentage of free iron ions is reduced to less than 0.005% of the overall amount of iron. Addition of N-methyl-D-glucamine (meglumine) and/or 2-amino-2-(hydroxymethyl)-1,3-propanediol (trometamol) causes further reduction in toxicity.

In this way, stabilized superparamagnetic single-domain particles with a mean particle diameter d₅₀ of 2 to 4 nm, preferably less than 3.5 nm, i.e., having a mean hydrodynamic particle diameter of from 5 to 8 nm, can be produced. Thus, the relaxivity ratio R2/R1 can be reduced to values between 1 and 3, preferably to 1-2. “Mean particle diameter d₅₀” means that at least 50% of the particles are in the specified diameter range. The particle size is determined using a Zetasizer from Malvern and an electron microscope. The mean particle diameter refers to particles having a hydrate envelope (Zetasizer) and those having no hydrate envelope (electron microscope).

Surprisingly, it was found that very small superparamagnetic single-domain particles with a particle diameter of less than 3.5 nanometers can pass through the kidneys, i.e., they are also suitable for MR diagnostics of the urinary tract and, above all, for molecular imaging.

In this way, the blood half-life of the very small superparamagnetic single-domain particles of the invention is substantially prolonged compared to previous particles, expanding the possible fields of use, e.g. in T1-weighted MR tomography used in angiography, lymphography and diagnosis of thrombi and tumors.

In a preferred embodiment of the production process according to the invention, the resulting aqueous dispersion is added with compounds containing polyamino groups so as to create possible ways of binding biologically active substances.

Binding of the compounds containing polyamino groups or of the purified reaction products of compounds containing polyamino groups with diagnostically effective substances, cell- and tissue-specific binding substances, pharmacologically active substances, pharmacologically active cells or cell fusion-mediating substances on the surface of the superparamagnetic single-domain particles stabilized against aggregation with acids may proceed via electrostatic interactions or covalent chemical binding as described above.

According to the invention, the dispersion can also be added with an iron-complexing agent, preferably glycerolphosphoric acid or a salt thereof.

The invention is also directed to a pharmaceutical composition comprising the above-defined inventive dispersion of stabilized and purified iron-containing particles, which optionally includes an iron-complexing agent and/or, optionally, positively charged metal ions and/or, optionally, compounds containing polyamino groups. The pharmaceutical composition may include pharmaceutically acceptable adjuvants such as sugars, preferably mannitol, sorbitol, glucose or xylitol. The sugars are included in amounts ensuring physiological conditions, e.g. an osmolality in the range of from 200 to 2,000 mOs/kg, preferably about 300 mOs/kg. For example, the pharmaceutical composition includes about 6% mannitol.

The invention is also directed to the use of the inventive aqueous dispersion in accordance with claims 20 to 23.

The main uses of the inventive dispersion containing very small superparamagnetic single-domain particles are in the field of MRT contrast media used in angiography, lymphography, diagnostics of thrombi and tumors, tumor damage, thrombolysis, immune enhancement, mediation of cell fusion, or in gene transfer, and here as well, the effectiveness of tumor damage, thrombolysis, cell fusion and gene transfer can be investigated using MRT diagnostics.

The dispersion according to the invention, which contains the very small stabilized superparamagnetic single-domain particles coated with compounds containing polyamino groups, such as pentaethylenehexamine, can be used in tumor diagnostics because accumulation can be observed in some tumor types upon injection into the bloodstream. When coupling pharmacologically active substances to the stabilized, very small superparamagnetic single-domain particles, the concentration thereof at the site of action can be increased, particularly in the event of very small superparamagnetic single-domain particles stabilized with cytostatic agents, such as doxorubicin or paclitaxel, bound to a polyamine such as pentaethylenehexamine, or when using tumor-specific antibodies. This fact is important in cancer therapy because the substances used in the chemotherapy of tumors cause very strong side effects throughout the organism and, if accumulation at the site of action takes place, the other regions of the body are less affected by cytostatic agents.

In animal experiments, the dispersion of the invention was found to have good effects as parenteral positive contrast medium in T1-weighted MR tomography, e.g. in the blood circulation, in diagnostics of thrombi and tumors, gastrointestinal tract imaging, and as antibody-specific contrast medium, where the long blood half-life has a positive effect because the reticuloendothelial system absorbs the particles only gradually, and the particles, particularly when coupled to antibodies, can move freely in the blood-stream for a prolonged period of time, thereby allowing increased accumulation at the binding site.

In T2-weighted MR tomography, the dispersion according to the invention still provides good negative contrast for liver, spleen, bone marrow and lymph nodes.

The amount of the inventive very small superparamagnetic single-domain particles is about 0.1 to 100 μmol Fe/kg body weight in uses as parenteral contrast medium in MRT and about 1 to 50 μmol Fe/kg body weight in uses as oral contrast medium in MRI.

The amount of the inventive very small superparamagnetic single-domain particles with bound radioactive metal ions is about 0.1 to 60 μmol Fe/kg body weight when used as parenteral contrast medium for MRT in combination with scintigraphy, SPECT or PET. The dose of bound radioactive metal ions such as technetium is between 150 and 300 MBq per patient in myocardial perfusion and between 100 and 220 MBq with gallium-67 citrate in scintigraphy of inflammatory diseases.

The inventive particles and the aqueous dispersion according to the invention are excellently suited for vascular diagnostics as positive and negative MR contrast medium in magnetic resonance tomographic assessment of lumen, wall and morphologic characterization of stenoses or obstructions of vessels (arteries, veins) of the body trunk, extremities, head-neck region, including intracranial vessels, of vessels close to the heart and coronary vessels, for assessing the microcirculation, including angiogenesis in the context with inflammatory diseases, infectious diseases or tumors, in the diagnostics of arterial walls affected by inflammation, including various stages of arteriosclerosis, for morphologic assessment of thrombi or emboli.

As explained in the examples below, the dispersion according to the invention can also be used with advantage in the diagnostics of primary tumors and metastases thereof and in the diagnostics of the lymphatic system, including detection of the sentinel lymph node.

It was found that the dispersion according to the invention can also be used in parenteral iron replacement therapy. To this end, a patient is administered i.v. with e.g. 20 μmol of iron per week and kg body weight. The particles accumulate in the liver and in organs of the hemopoietic system (bone, marrow, spleen) and, depending on the particle size, are released into the blood only gradually (sustained release) over days or weeks, so that a depot effect is achieved.

Owing to the very good tolerability and long-term circulation of the purified and formulated iron oxide particles of the invention included in the dispersion, uses in tumor therapy following intravenous, intraarterial and intratumoral injection in combination with magnetic fields (magnetic field hyperthermia), embolizates and chemotherapeutic agents are possible. In this way, increased accumulation in the target tissue by binding of so-called target-specific ligands on the iron oxide particles can be achieved. FIG. 3.1 illustrates the increase of intratumoral accumulation of the particles by polyamine binding on the surface and, as a consequence, targeting towards angiogenic endothelium.

Therefore, the invention is also directed to said superparamagnetic iron-containing particles of iron hydroxide, iron oxide hydrate, iron oxide, iron mixed oxide or iron with a particle size of from 2 to 10 nm, which particles bear aliphatic di- and/or tricarboxylic acids selected from citric acid, malic acid, tartaric acid, derivatives or mixtures thereof as stabilizer substances on their surface, which particles are characterized in that they have a content of free iron ions below 0.005% of the overall amount of iron and can be produced by precipitation of the iron-containing particles from aqueous iron salt solutions using an alkali solution or ammonium hydroxide, subsequent treatment with said aliphatic di- and/or tricarboxylic acids or mixtures thereof, purification of the particles thus stabilized using dialysis with distilled water until the dialyzate has an electric conductivity of less than 10 μS/cm, subsequent treatment of the dialyzate with aqueous salt solutions of aliphatic di- and/or tricarboxylic acids selected from citric acid, malic acid and tartaric acid, dialysis with distilled water until the dialyzate has an electric conductivity of less than 10 μS/cm, and subsequent treatment of the dialyzate with aqueous solutions of free di- and/or tricarboxylic acids selected from citric acid, malic acid, tartaric acid and dialysis with distilled water until the dialyzate has an electric conductivity of less than 10 μS/cm and the content of free iron ions is less than 1 mg/l, addition of N-methyl-D-glucamine (meglumine) and/or 2-amino-2-(hydroxymethyl)-1,3-propanediol (trometamol) to the dispersion, and isolation of the iron-containing particles from the prepared dispersion.

Consequently, the inventive iron particles with a content of free iron ions of <0.005% of the overall amount of iron are characterized in that the aliphatic di- and/or tricarboxylic acids or mixtures thereof, which the particles bear as stabilizer substances on the surface thereof, have N-methyl-D-glucamine (meglumine) and/or 2-amino-2-(hydroxymethyl)-1,3-propanediol (trometamol) as cations.

### EXAMPLES

### Preparative Example 1

### Comparative Example

Iron(III) chloride (270 g) and iron(II) chloride (119 g) are dissolved in 1 l of distilled water with stirring and heated to 80° C. with exclusion of oxygen. The pH value of the solution is adjusted to 10 by adding aqueous ammonia with stirring. Thereafter, the dispersion is cooled to about 60° C., adjusted to pH 7.0 with citric acid and dialyzed with distilled water until the dialyzate has an electric conductivity of <10 μS/cm. To remove fairly large or slightly aggregated superparamagnetic particles, the dispersion is centrifuged at 10,000 rpm for 10 min. The centrifuged material of the dispersion is removed, placed in an ultrafiltration apparatus with 5 kD filter and dialyzed with distilled water until the dialyzate has an electric conductivity of less than 10 μS/cm. The conductivity was determined using a conductivity measuring instrument from the Knick Company.

The prepurified dialyzate can be used as starting dispersion to produce a positive i.v. contrast medium for MRT diagnostics.

### Preparative Example 2

### Comparative Example

Iron(III) chloride (270 g) and iron(II) chloride (119 g) are dissolved in 1 l of distilled water with stirring and heated to 80° C. with exclusion of oxygen. The pH value of the solution is adjusted to 10 by adding aqueous ammonia with stirring. Thereafter, the dispersion is cooled to about 60° C., adjusted to pH 7.0 with citric acid, and the dispersion is adjusted to a conductivity of 150 mS/cm using distilled water. Subsequently, the dispersion is placed on a magnet with a magnetic flux density of 0.1 T for 5 hours. The supernatant of the dispersion is removed and dialyzed with distilled water until the dialyzate has an electric conductivity of <10 μS/cm.

The prepurified dispersion is added with 50 ml of a 20 wt. % solution of trisodium citrate in distilled water, filled up to 1 l with distilled water and dialyzed. This process is repeated until the content of free iron ions is less than 1 mg of iron/I.

### Preparative Example 3

A prepurified dispersion having an electric conductivity of <10 μS/cm is prepared as in Example 2.

The dispersion is added with 50 ml of a 20 wt. % solution of disodium hydrogen citrate in distilled water, filled up to 1 l with distilled water and dialyzed. This process is repeated until the content of free iron ions is less than 1 mg of iron/I.

Thereafter, the dispersion is adjusted to a pH value of 5 using a 20 wt. % solution of citric acid and dialyzed with distilled water until the content of free iron ions is less than 1 mg of iron per liter.

### Preparative Example 4

100 ml of the dispersion of very small superparamagnetic single-domain particles of Example 3, having an iron content of about 200 g of iron/I, is adjusted to a pH value of 7.5 using a 1 M solution of D-(−)-N-methylglucamine in distilled water. This dispersion is used to produce a galenic formulation of an MR contrast medium.

### Preparative Example 5

A quantity of dispersion according to Example 4, containing 2.79 g of iron, is placed in a 100 ml volumetric flask. This is added with 6 g of mannitol and 0.304 g of sodium glycerophosphate dissolved in 50 ml of distilled water and filled up to make 100 ml. The resulting galenic formulation is sterile-filtrated into a 100 ml ampoule through a 0.2 μm filter and the ampoule is heat-sterilized at 121° C. The cooled dispersion can be used as MRT contrast medium in angiography, lymphography, diagnostics of thrombi and tumors.

### Preparative Example 6

Iron(III) chloride (270 g) and iron(II) chloride (119 g) are dissolved in 1 l of distilled water with stirring and heated to 85° C. with exclusion of oxygen. The pH value is adjusted to 10.5 by dripping a 25% ammonium hydroxide solution. Immediately after precipitation, the dispersion is adjusted to a pH value of 7.0 using a solution of 25 g of citric acid and 25 g of tartaric acid in 500 ml of water and stirred at 85° C. for 20 min. Thereafter, the dispersion is cooled to about 20° C., adjusted to a pH value of 7.0 using hydrochloric acid, added with 20 ml of 30% hydrogen peroxide and stirred until gas evolution ceases. The dispersion is dispersed for 20 min using ultrasound of 300 W power and subsequently dialyzed until the dialyzate has an electric conductivity of less than 10 μS/cm. The dispersion is centrifuged at 10,000 rpm for 10 min to remove fairly large or slightly aggregated superparamagnetic particles.

### Preparative Example 7

100 ml of the prepurified dispersion of very small superparamagnetic single-domain particles of Example 6, having an iron content of about 100 g iron/I, is added with 50 ml of a 20 wt. % solution of sodium dihydrogen citrate in distilled water, filled up to 1 l with distilled water and dialyzed. This process is repeated until the content of free iron ions is less than 1 mg of iron/I. Thereafter, the dispersion is adjusted to a pH value of 5 using a 20 wt. % solution of citric acid and dialyzed with distilled water until the content of free iron ions is less than 1 mg of iron per liter.

### Preparative Example 8

100 ml of the purified dispersion of very small superparamagnetic single-domain particles of Example 7, having an iron content of about 100 g of iron/I, is adjusted to pH 6.0 using a 0.1 M solution of pentaethylenehexamine in distilled water and subsequently to pH 7.5 using a 1 M solution of D-(−)-N-methylglucamine in distilled water. This dispersion is used in coupling of diagnostically effective substances, cell- and tissue-specific binding substances, pharmacologically active substances, pharmacologically active cells or cell fusion-mediating substances.

### Preparative Example 9

A quantity of dispersion according to Example 8, containing 2.79 g of iron, is placed in a 100 ml volumetric flask. This is added with 6 g of mannitol and 0.304 g of sodium glycerophosphate dissolved in 50 ml of distilled water and filled up to make 100 ml. The resulting galenic formulation is sterile-filtrated into a 100 ml ampoule through a 0.2 μm filter and the ampoule is heat-sterilized at 121° C. The cooled dispersion can be used as MRT contrast medium in angiography, lymphography, diagnostics of thrombi and tumors, and more advantageously in the diagnostics of prostate tumors.

### Preparative Example 10

A solution of 65 mg of fluorescein isothiocyanate in 10 ml DMF is mixed with a solution of 950 mg of pentaethylenehexamine in 10 ml of DMF. An orange-colored precipitate is obtained. After one hour, the precipitate is dissolved in water and added to 100 ml of the dispersion of very small superparamagnetic single-domain particles of Example 7, which has an iron content of about 100 g of iron/I. Subsequently, the dispersion is adjusted to pH 7.5 using a 1 M solution of trometamol (2-amino-2-(hydroxymethyl)-1,3-propanediol) in distilled water. Using a 50 kD filter, the mixture is filled in an ultrafiltration apparatus and dialyzed with distilled water until the dialyzate has an electric conductivity of less than 10 μS/cm. The purified dispersion can be used to produce an MRT contrast medium for angiography, diagnostics of thrombi and tumors, more advantageously for diagnostics of sentinel lymph nodes in cases of mammary carcinoma, and for labeling living cells.

Typical analytical data of the very small superparamagnetic single-domain particles are as follows:

| | | |
|---|---|---|
| Particle diameter d₅₀: | 3.8 | nm |
| Overall diameter with stabilizer: | 9 | nm |
| T1 relaxivity: | 20 | l/mmol s |
| T2 relaxivity: | 38 | l/mmol s |

| | |
|---|---|
| Relaxivity ratio R2/R1: | 1.9 |

1) Influence of Preparation, Purification, Formulation and Heat-Sterilization on the Tolerability in Rats Following Intravenous Injection of Samples

The tolerability was investigated in rats (male, Wistar, 200 to 250 g), using the non-toxic dose level as parameter. This is the dose where none of the rats from a test group (n=3 animals per dosage group) had died within two weeks following intravenous injection of a sample.

The influence of intravenous injection of a sample on protein excretion and hemoglobin excretion (hemolysis) was investigated in another test. To this end, the rats, having received an intravenous injection of sample, were placed in a plastic box cleaned with distilled water, and the discharge of spontaneous urine was observed. The spontaneous urine was investigated each hour for four hours following injection. The spontaneous urine was collected within a few seconds after discharge and applied to a urine test strip (Combistix®, Bayer AG). The protein and hemolysis pads were read within the period of time prescribed by the manufacturer.

The results of the investigations are illustrated in Table 1. As can be seen, the stabilized and prepurified sample produced according to Example 1 (WO 97/35200), which was merely added with 6% mannitol, was extremely intolerable after heat sterilization, and the animals died even at very low dosages, rendering measurement of the renal physiology impossible.

A reduction in toxicity is achieved by treating the aqueous dispersion of citric acid-stabilized and prepurified superparamagnetic single-domain particles with solutions of tri-, di- and mono-salts of citric acid and subsequently dialyzing with distilled water (cf. Example 2).

Further improvement in tolerability is achieved by the inventive purification using citric acid salts (of Example 2) and adjusting a pH value of about 5 with free citric acid and subsequent dialysis with distilled water (cf. Example 3). Further reduction in toxicity is achieved by neutralizing the dispersion thus formed with a solution of D-(−)-N-methylglucamine in distilled water (cf. Example 4).

By formulating the galenic preparation with the sodium glycerophosphate complexing agent (cf. Example 5), dosages of up to 3 mmol Fe/kg as bolus injection are well tolerated by the rats without side effects (proteinuria, hemolysis).

TABLE 1
Influence of purification and galenic formulation on the tolerability of samples following intravenous injection in rats

| | | | Example 4 | Example 5 |
|---|---|---|---|---|
| | Example 1 | Example 2 | purified as in Example 3 | purified as in Example 4 |
| | prepurified | purified, Na citrate | methylglucamine | with Na glycerophosphate |
| | unformulated | unformulated | unformulated | formulated |
| | heat-sterilized | heat-sterilized | heat-sterilized | heat-sterilized |
| Non-toxic | >0.1 mmol Fe/kg | up to 1 mmol Fe/kg | up to 2 mmol Fe/kg | up to 3 mmol Fe/kg |
| dose level | | | | |
| Hemoglobinuria | no urine obtainable be- | from 0.5 mmol Fe/kg on | from 1.5 mmol Fe/kg on | from 2 mmol Fe/kg on |
| | cause toxic dose very low | | | |
| Proteinuria | no urine obtainable be- | from 0.5 mmol Fe/kg on | from 1.5 mmol Fe/kg on | from 2 mmol Fe/kg on |
| | cause toxic dose very low | | | |

Non-toxic dose level: the dose where none of the rats from a test group had died within two weeks following injection.

As demonstrated by the results in rats, the purification steps and galenic additives furnish a biologically applicable product, and no toxic effects appear upon intravenous injection, even at very high dosages of up to about 100 times the clinically required dose.

2) Influence of Preparation, Purification, Formulation and Heat-Sterilization on the Effectiveness in Rats Following Intravenous Injection of Samples

The blood circulation time of samples from Examples 1, 2 and 5 was determined in male Wistar rats (200-250 g) by measuring the magnetic effect. To this end, the samples were injected intravenously at a dose of 0.05 mmol Fe/kg. Blood was collected prior to and 1, 2, 5, 10, 15, 20, 30, 60, 90, 180 and 240 min after injection of the samples. The relaxation time (longitudinal and transversal relaxation time) in the blood was measured by means of relaxometry at 0.94 T (Bruker Minispec, Bruker, Karlsruhe, Germany). An effect-time profile was established on the basis of the time (following injection) and relaxation times in the blood.

The blood half-life was calculated by adapting these data to a simple exponential function. The blood half-life is a pharmacokinetic parameter describing the clearance of an active substance from the blood. The longer the circulation of the active substance in the blood, the longer the blood half-life.

TABLE 2
Influence of purification and galenic formulation on the effectiveness
of samples following intravenous injection in rats

| | | | Example 4 | Example 5 |
|---|---|---|---|---|
| | Example 1 | Example 2 | purified as in Example 3 | purified as in Example 4 |
| | prepurified | purified, Na citrate | methylglucamine | with Na glycerophosphate |
| | unformulated | unformulated | unformulated | formulated |
| | heat-sterilized | heat-sterilized | heat-sterilized | heat-sterilized |
| Blood half-life | 5 min | 10 min | 20 min | 40 min |

Surprisingly, the results show that the inventive purification and formulation with sodium glycerophosphate also extends the residence time of the particles in the blood. This contradicts tolerability because one might tend to assume that particles which have a long residence time in the blood and do not undergo rapid elimination might develop a toxic effect.

For use as diagnostic agent or in therapy, long blood half-life is advantageous because in this way, higher concentrations in the target tissue can be achieved before the particles are cleared out of the blood.

Owing to the adequate tolerability of the particles and long circulation in the blood, the formulations according to the invention can be used in medical diagnostics and therapy with advantage.

### Use Example 1

### Vascular Diagnostics

The particles can be used as positive (brightening) and negative (darkening) contrast media in magnetic resonance tomographic assessment of lumen, wall and morphologic characterization of stenoses or obstructions of vessels (arteries, veins) of the body trunk, extremities, head-neck region, including intracranial vessels, of vessels close to the heart and coronary vessels. This can be done following intravenous and intraarterial bolus injection.

Bolus angiography in a pig (FIG. 1.1) and equilibrium angiography of the coronary vessels in a healthy subject (FIG. 1.2) are illustrated with the aid of exemplary figures.

Owing to the good tolerability of the particles, bolus injection is also possible in humans. The long circulation time allows high-resolution imaging of the coronary vessels in humans (FIG. 1.2).

Apart from assessing large vessels, the iron oxide nanoparticles of the invention allow assessment of the microcirculation, including angiogenesis in the context with inflammatory diseases, infectious diseases or tumors. As an example, FIG. 1.3 illustrates the myocardial perfusion in a pig, with underperfusion in an artificially generated myocardial infarction (FIG. 1.3).

Accumulation of the inventive particles in arterial walls affected by inflammation (various stages of arteriosclerosis) allows early recognition of infarction risks, irrespective of the extent of a vascular stenosis. This is illustrated in Exemplary FIG. 1.4. Furthermore, the particles can be used in morphologic assessment of thrombi or emboli (arterial or venous).

### Use Example 2

### Diagnostics of Tumors and Metastases Thereof, Including Pathways of Metastasization in the Lymphatic System

The well-tolerable, purified and formulated iron oxide particles of Example 5 are used in MRT diagnostics of primary tumors and metastases thereof. This can be done using T1-weighted imaging (brightening effect, FIG. 2.1) and T2-weighted imaging (darkening effect, FIG. 2.2). Exemplary FIG. 2.1 illustrates the use for improved representation of a liver tumor in a rat.

The magnetic properties and good tolerability of the purified and formulated particles of the invention allow injection into the lymphatic system or regions (body tissue, organs, tumors) from where tissue fluid (lymph) is transported to a lymph node. In tumor diagnostics, this is utilized to detect the sentinel lymph node. This is the crucial lymph node which, as the first possible lymph node, receives metastases from a primary tumor. Possible metastatic affection of the sentinel lymph node is decisive in therapy planning and prognosis of tumor diseases. Injection of the iron oxide nanoparticles of Example 5 into the region of the primary tumor and T1-weighted imaging allows assessment of the lymphatic vascular system (brightening magnetic effect of the iron oxide nanoparticles). When using T2-weighted MRT imaging, it is possible to assess the lymph nodes and possible metastases (darkening magnetic effect of the iron oxide nanoparticles). This is illustrated in Exemplary FIG. 2.3. Using additional binding of dyes (visual, fluorescence technique) or binding of radioactive substances (technetium, indium), the assessment of the lymphatic system can be combined into an MRT-optical imaging or MRT-nuclear-medical imaging.

### Use Example 3

### Tumor Therapy

Owing to the very good tolerability and long circulation time of the purified and formulated iron oxide particles of the invention, use in tumor therapy is possible following intravenous, intraarterial and intratumoral injection in combination with magnetic fields (magnetic field hyperthermia), embolizates and chemotherapeutic agents. In this way, increased accumulation in the target tissue by binding of so-called target-specific ligands on the iron oxide particles can be achieved. Exemplary FIG. 3.1 illustrates the increase of intratumoral accumulation of the particles by polyamine binding on the surface and, as a consequence, targeting towards angiogenic endothelium. In addition, the non-modified particles, owing to their long intravasal residence time, allow detection of the mircocirculation of tumors and in this way possible therapeutic effects in the context with a tumor therapy (therapy monitoring).

### Use Example 4

### In Vivo Cell Monitoring

Using the well-tolerable purified and formulated iron oxide particles, it is possible to label cells (stem cells, endothelial cells, dendritic cells, organ cells, immune cells) outside the body in such a way that cells are incubated e.g. with a dispersion of Example 3 for 30 to 60 min and washed. After injection of these labeled cells into the body (intravenous, intraarterial, lymphatic, into tissues, organs or pathological processes), the cells can be monitored within the living body. As an example, the representation of neuronal stem cell labeling is demonstrated in a rat Parkinson model (FIGS. 4.1 and 4.2).

By binding dyes or radioactive markers, it is possible to combine MRT and optical imaging or to combine MRT and nuclear-medical imaging such as scintigraphy, SPECT or PET in order to investigate the morphology, function and biochemistry of cells labeled with superparamagnetic particles in a living organism.

Receptor Imaging for the Diagnostics of Inflammatory Processes

Inflammatory processes in the human body cause accumulation of autologous immune cells such as macrophages. The macrophages absorb the superparamagnetic nanoparticles, and the inventive particles of Example 5 accumulate in the inflamed regions. Such “magnetizable macrophages” can be made visible in T2-weighted images in an MR tomograph. Fields of use include rheumatism, for example.

Arteriosclerosis

Accumulation of magnetic particles from the sample of Example 5 in an arteriosclerotic plaque gives rise to an effect that reduces the T2 relaxation time, with signal loss in the vessel wall. The arteriosclerotic plaques are represented in a dark contrast. Accumulation of magnetic particles of this sample indicates the presence of inflammatory cells and macrophages in the arteriosclerotic plaque.

MRT of Neurodegenerative Diseases

In many neurodegenerative diseases, such as Alzheimer's disease or multiple sclerosis, augmented apoptosis is of eminent importance. Initial tests with very small particles of Example 4 (particle diameter 3.5 nm) in mice having a passive experimental autoimmune encephalomyelitis (EAE) show accumulation of the particles in cortex regions affected by multiple sclerosis (MS).

By binding dyes or radioactive markers, it is possible to produce MR contrast media for parenteral use, enabling a combination of MRT and optical imaging or a combination of MRT and nuclear-medical imaging such as scintigraphy, SPECT or PET.

Monitoring of Therapy-Related Apoptosis e.g. in Tumor Therapy with Annexin V-Coupled Particles of the Invention

To date, the success of a tumor therapy has been predominantly rated on the basis of morphologic criteria which, however, can usually be established only after weeks or even months. In contrast, in vivo imaging of apoptosis can aid in early or simultaneous monitoring of therapeutic success because induction of apoptosis proceeding prior to the resulting tumor regression is initiated within hours or a few days. In vivo imaging of these early transformations can substantially reduce the time required for assessing therapeutic success, ultimately allowing therapeutic concepts to be modified at a substantially earlier point in time, if necessary. In this way, precious time to increase the prospects of treatment can be gained and excessive side effects, but also, expenses for an ineffective therapy can be reduced. Ideally, apoptosis imaging can provide real-time information as to the spatial distribution of apoptosis and consequently allow informative characterization of pathological processes in a variety of pathological conditions.

Detection of Thrombosis Using MRT

Using the particles according to the invention, it is possible to detect acute thrombi by means of MRT, as demonstrated by investigations on rats and rabbits.

Therapy of Inflammatory Plaques

Accumulation of magnetic particles from the sample of Example 5 in an arteriosclerotic plaque gives rise to an effect that reduces the T2 relaxation time, with signal loss in the vessel wall. The arteriosclerotic plaques are represented in a dark contrast. Coupling of anti-inflammatory substances, such as paclitaxel or matrix metalloproteinase inhibitors (MMP) such as marimastat, neovastat, sirolimus or tacrolimus, to the particles according to the invention results in accumulation of these anti-inflammatory substances in the inflammatory plaques and consequently in inhibition of inflammation.

Transfection Vehicle in Gene Therapy

It was found in experiments that particles according to the invention with polyamine-coated surfaces according to Example 8 are suitable as in vitro transfection agents for DNA and RNA in cell cultures of colon carcinomas. Accumulation of DNA and RNA bound to the magnetic particles gives rise to an effect in the cells that reduces the T2 relaxation time, with signal loss in the cells, and thus can be used as a measure of transfection success.

Adjuvant in Immune Enhancement Towards Viruses, Bacteria and Tumor Cells

Therapeutic tests using conjugates of the inventive particles according to Example 8 with cell wall components of tumor cells were carried out on implanted prostate and colon carcinomas in animal experiments. An immune reaction resulting in tumor necrosis was observed.

### LEGENDS TO THE FIGURES

FIG. 1.1: Magnetic resonance tomographic representation of the renal arteries in a pig during bolus injection of the sample of Preparative Example 5 (arterial MRT bolus angiography). MRT bolus angiography of the renal arteries and aorta in a pig at 1.5 Tesla using T1-weighted gradient echo technique, repetition time 6 ms, echo time 1.7 ms, excitation angle 25°. A dose of 0.025 mmol Fe/kg of the sample of Preparative Example 5 was injected intravenously in the form of a rapid bolus. As a result of contrast medium arrival in the arterial vessels, an angiographic image of the vessels without representation of veins is obtained. It is precisely the good tolerability of the sample of Example 5 that makes bolus injection possible. The high magnetic efficiency results in a substantial reduction of the T1 relaxivity of the blood, which in turn results in a bright representation of the vessels.

FIG. 1.2: Magnetic resonance tomographic representation of coronary vessels (MRT coronary angiography) in a human after injection of a sample of Preparative Example 5. Injection of the sample improved the representation of the coronary vessels in a human. A healthy subject was examined at 1.5 Tesla with a gradient echo technique with a repetition time of 4.5 ms, an echo time of 1.7 ms and an excitation angle of 25° prior to and after injection of the sample at a dose of 0.045 mmol Fe/kg. A section of the right coronary artery of a healthy subject is marked with an arrow. Prior to administering the contrast medium ((A) on the left in the Figure), the represented section of the right coronary artery has poor definition and shows interruptions. When using the contrast medium according to the invention, the vessel section is seen with high definition and rich in contrast. Likewise, the ventricles are represented very brightly, allowing good differentiation from the cardiac muscles. The contrast is retained over a period of up to 50 minutes following injection, allowing a high-definition representation of the entire vascular system of the heart. In FIG. 1.2 (B), a three-dimensional reconstruction of the coronary vessels from the measured single layers was performed. This is only possible owing to the high effectiveness and long blood residence time of the inventive sample.

FIG. 1.3: Microcirculation in healthy heart tissue compared to infarction in a pig in the equilibrium phase. Representation of the microcirculation exemplified in an artificially generated infarction in a pig. The MRT examination was performed at 1.5 Tesla using an electrocardiographically triggered gradient echo technique with a repetition time of 5 ms, an echo time of 2 ms and an excitation angle of 25°. This is an examination during the equilibrium phase. The sample of Preparative Example 5 has a long blood residence time, developing the effect of magnetic contrast therein, so that the heart muscle tissue with good blood circulation is represented as bright compared to the dark infarction area (B) with poor blood circulation at the lower edge of the left heart muscle represented in the form of a circle. Reliable differentiation from the infarction is not possible without contrast medium (A).

FIG. 1.4: Representation of the vessel wall morphology with inflammation in a Watanabe rabbit with hereditary hyperlipidemia used as a model of arteriosclerosis (double-contrast MRT angiography in the rabbit for arteriosclerotic plaque representation). Examinations were performed on rabbits, using a clinical MR tomograph at 1.5 Tesla with a moderately T1-weighted gradient echo technique with a repetition time of 100 ms, an echo time of 3.2 ms and an excitation angle of 25°. Prior to intravenous injection of the sample of Preparative Example 5, the central cervical vessels are dark in the representation, while the vessel wall appears brighter (A and enlarged detail C). After injection of the sample, the representation of the vessel lumen is brighter, i.e., including more signals, as a result of the effect of the sample which reduces the T1 relaxation time (B and enlarged detail D). The cervical vessels are shown by the white arrow heads in D. Accumulation of magnetic particles from the sample in the arteriosclerotic plaque gives rise to an effect that reduces the T2 relaxation time, with signal loss in the vessel wall. The arteriosclerotic plaques are represented as a dark contrast. Accumulation of magnetic particles of the sample demonstrates the presence of inflammatory cells and macrophages in the arteriosclerotic plaque. The magnetic properties of the sample particles allow investigation of vessels with double contrast. The healthy vascular lumen appears bright as a result of the freely circulating particles, and dangerous vessel wall transformations appear in dark or blackened representation. The accumulation of the iron-containing magnetic particles in the section preparation (F) and in the histological section (E) of the aorta of this rabbit can be represented macroscopically using the Berlin blue iron reaction (accumulated iron is blue). Histologically, the accumulation of magnetic particles of the sample can be seen in the macrophages of the arteriosclerotic plaque (E). Macrophages represent inflammatory and therefore dangerous transformations in the vessel wall, which may lead to sudden myocardial infarction because tearing of the vessel wall may occur at this position, giving rise to formation of a thrombus.

FIG. 2.1: Magnetic resonance tomographic representation of a liver tumor (implanted colon carcinoma CC531) in a rat in T1-weighted imaging with positive contrast. Examination in frontal layer orientation at 1.5 Tesla using a T1-weighted gradient echo sequence with a repetition time of 6.8 ms, an echo time of 2.3 ms and an excitation angle of 25°. Prior to injection of contrast medium (A), differentiation of the tumor in the liver is difficult. After injection of the sample of Preparative Example 5 at a dose of 0.03 mmol Fe/kg KGW (B), the representation of the liver is very bright and signal-rich. The dark tumor at the bottom edge has distinct boundaries. The upper large part of the tumor and the lower small part are recognized very clearly.

FIG. 2.2: Magnetic resonance tomographic representation of a liver tumor (implanted colon carcinoma CC531) in a rat in T2-weighted imaging with negative contrast. Examination in axial layer orientation at 1.5 Tesla using a T2-weighted gradient echo sequence with a repetition time of 200 ms, an echo time of 12 ms and an excitation angle of 12°. Prior to injection of contrast medium (A), the liver is very bright and the tumor in the liver cannot differentiated. The stomach filled with feed and air (on the right in FIG. A) appears in a dark representation. After injection of the sample of Preparative Example 5 at a dose of 0.03 mmol Fe/kg KGW (B), the liver is represented black as a result of the effect of the magnetic particles which reduces the T2 relaxation time. The signal-rich (bright) tumor at the upper edge of the liver is now clearly visible.

FIG. 2.3: Magnetic resonance tomographic lymphography and lymphangiography in a rat in T1- and T2-weighted imaging. The examinations were performed at 1.5 Tesla in frontal layer orientation. A sample of Example 5, 0.02 ml of solution including 0.02 mmol of iron per ml, was injected into the right hindpaw of a rat. In the T1-weighted measuring technique (A) using a gradient echo technique with a repetition time of 50 ms, an echo time of 5 ms and an excitation angle of 250, the lymphatic vessel can be recognized, which transports (small arrow heads) the lymph from the site of injection (paw) to the sentinel lymph node (in bright representation). The small arrow points to the lymph node. The bright lymph in the marginal sinus of the lymph node is represented therein. Examination was performed about 5 min following injection. In the T2-weighted gradient echo measurement (B) with a repetition time of 100 ms, an echo time of 11 ms and an excitation angle of 15°, the actual lymph node can be seen, wherein iron particles of the sample have accumulated. In this measuring technique, accumulation results in signal quenching (arrow).

FIG. 3.1: Magnetic resonance tomographic representation of angiogenesis targeting using the polyamine-modified single-domain particles of the sample of Preparative Example 8 in a rat prostate carcinoma Dunning tumor G cell line. The examination of the rats was performed at 1.5 Tesla in axial layer orientation using a T2-weighted gradient echo technique (repetition time 200 ms, echo time 15 ms, excitation angle 15°).

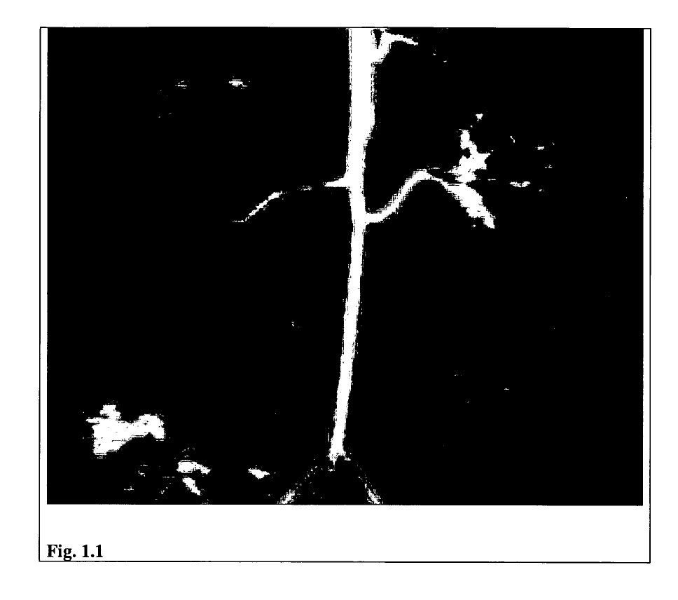

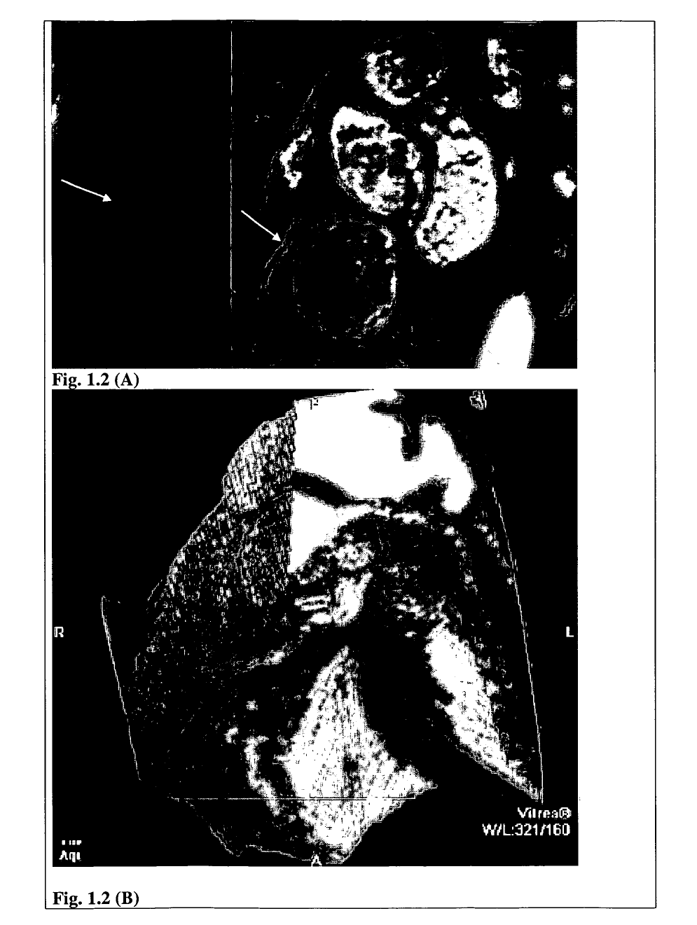

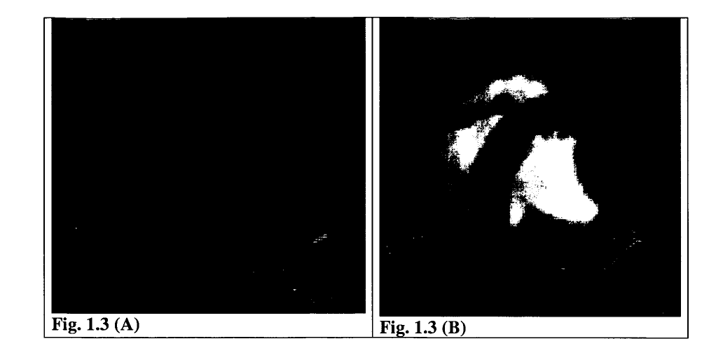

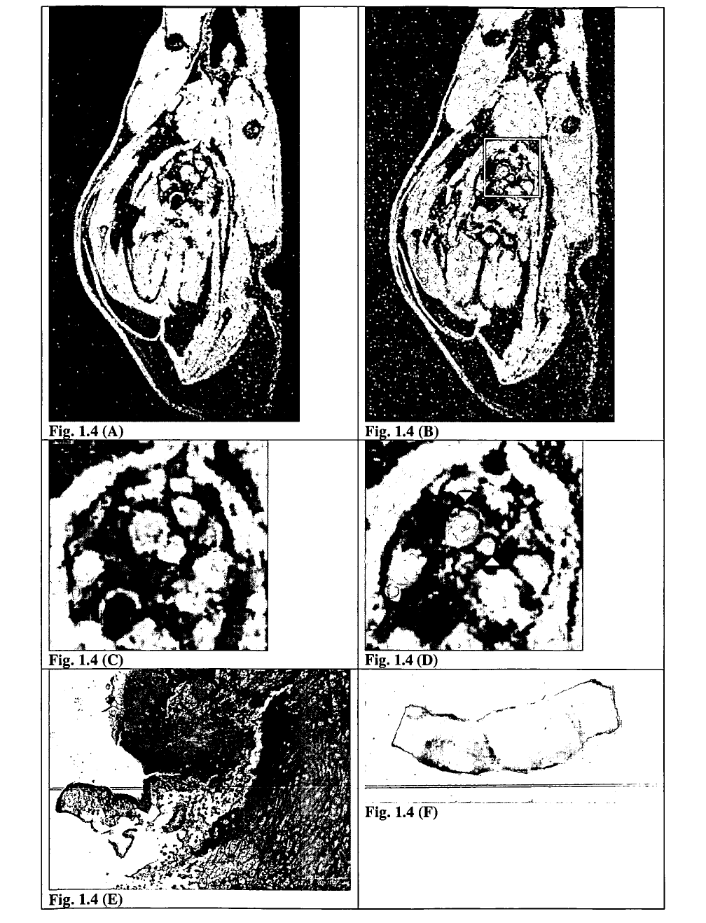

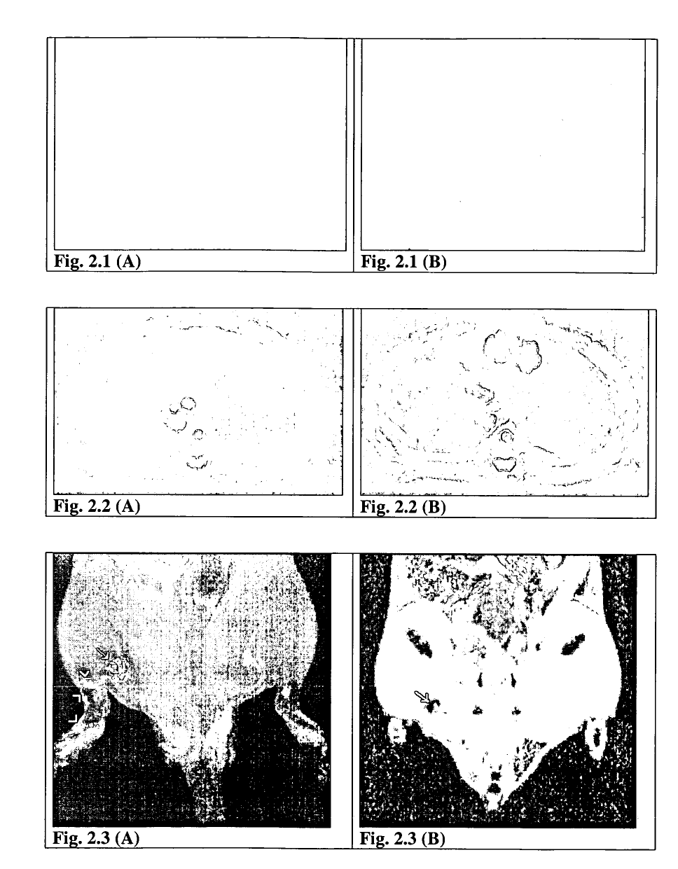

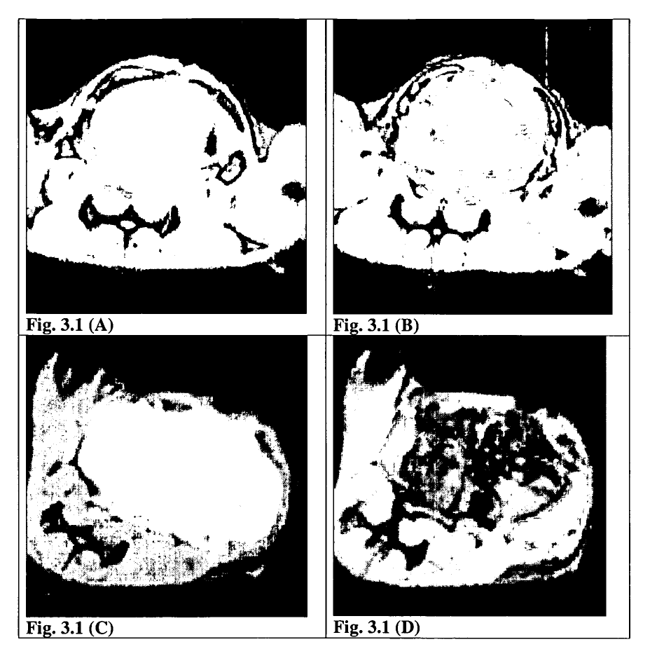

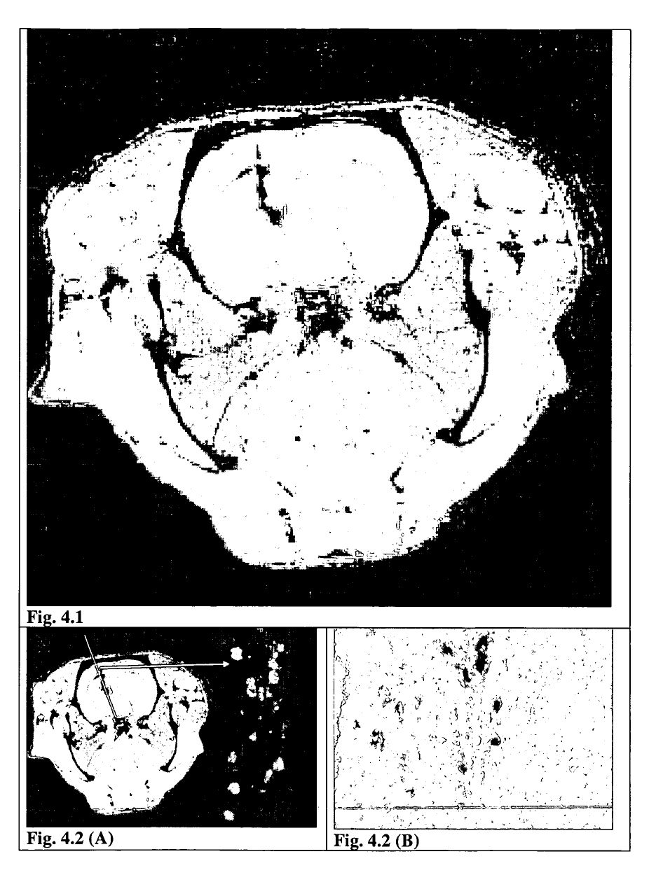

(A) and (C) are investigations prior to injection of the samples. The tumor is represented brighter compared to the surrounding tissue. After intravenous injection of the sample of Preparative Example 5 (B) at a dose of 0.045 mmol Fe/kg body weight, the tumor regions after injection are seen to be inhomogeneously brighter as well as darker compared to the image prior to intravenous injection of the sample (A), showing regions with high vessel density (brighter regions) and regions with accumulation in tumor tissue (darker regions). FIG. 3.1 (D) shows strong accumulation of particles in the tumor (dark) after intravenous injection of the sample of Example 8 at a dose of 0.045 mmol Fe/kg. The angiogenic vascular endothelium has high density on receptors for positive charges. Modification with amine renders the surface of the particles positive. Compared to the negative sample of Example 5, this results in very strong accumulation of the particles in tumor tissue, giving rise to a dramatic signal reduction (blackening, (D)) compared to the blank image (C).

FIG. 4.1: Magnetic resonance tomographic monitoring of neuronal stem cells in the brain of rats, which cells were previously labeled with iron oxide particles and subsequently implanted. Examinations were performed at 7 Tesla, using a T2-weighted gradient echo technique (repetition time 490 ms, echo time 5.4 and excitation angle 15°). MR tomographic representation of a rat 16 weeks after implantation of 100,000 neuronal stem cells incubated with the sample of Example 5 prior to implantation. A region with signal quenching (black) by cells labeled with the sample is recognized in the brain. Even after 16 weeks, the cells can be imaged at the site of implantation by means of MR tomography.

FIG. 4.2: Magnetic resonance tomographic monitoring of implanted neuronal stem cells in the brain of rats compared to fluorescence labeling (A) and iron staining (B). After completed MRT investigation, histological sections of the rat brain were prepared in an orientation along the puncture channel (A, white line). Prior to implantation and labeling with the sample of Example 5, the neuronal stem cells were transfected with a gene for the production of a green fluorescent protein. Histology reveals that the implanted cells remain alive even 16 weeks after implantation and produce the green fluorescent protein (A, arrow). This can be seen in a fluorescence-microscopic examination of the implantation channel. Berlin blue iron staining shows the iron (B, blue cells) of the sample according to the invention incorporated by the cells prior to implantation. The localization of the blue iron staining and green fluorescence shows good agreement.

1. An aqueous dispersion of superparamagnetic single-domain particles of iron hydroxide, iron oxide hydrate, iron oxide, iron mixed oxide or iron with a particle size of from 2 to 10 nm, which particles bear aliphatic di- and/or tricarboxylic acids selected from citric acid, malic acid, tartaric acid, derivatives or mixtures thereof as stabilizer substances on their surface, characterized in that the dispersion comprises N-methyl-D-glucamine (meglumine) and/or 2-amino-2-(hydroxymethyl)-1,3-propanediol (trometamol) and that the content of free iron ions is less than 1 mg/l.

2. The dispersion according to claim 1, wherein a physiologically tolerable complexing agent for iron ions is included.

3. The dispersion according to claim 1, wherein the physiologically tolerable complexing agent included in the dispersion is glycerophosphoric acid, ethylenediaminetetraacetic acid (EDTA), N-hydroxyethylethylenediaminetriacetic acid (HEDTA), diethylenetriaminepentaacetic acid (DTPA), α-mercaptopropionylglycine (thiopronine), 2,3-mercapto-1-propanesulfonic acid, 30-amino-3,14,25-trihydroxy-3,9,14,20,25-pentaazatriacontane-2,10,13,21,24-pentaone-methanesulfonic acid (deferoxamine mesylate), a mixture or salt thereof, the cations of the salts preferably being sodium, potassium, calcium, magnesium, D-(−)-N-methylglucamine (meglumine), 2-amino-2-(hydroxymethyl)-1,3-propanediol (trometamol) or mixtures thereof.

4. The dispersion according to claim 3, wherein the complexing agent is glycerophosphoric acid or a salt thereof.

5. The dispersion according to any of claim 1, wherein the single-domain particles consist of Fe₂O₃ or Fe₃O₄, iron mixed oxides of general formula mMO.nFe₂O₃, wherein M represents the bivalent metal ions Fe, Co, Ni, Mn, Be, Mg, Ca, Ba, Sr, Cu, Zn, Pt or mixtures thereof, or mixed oxides of general formula mFe₂O₃.nMe₂O₃, wherein Me represents the trivalent metal ions Al, Cr, Bi, rare earth metals or mixtures thereof, or iron, wherein m and n in each of the above formulas are integers of from 1 to 6.

6. The dispersion according to any of claim 1, wherein it includes physiologically tolerable compounds containing polyamino groups, selected from the group of polyethyleneimines (PEI), polyvinylamines (PVAm), PEI and PVAm copolymers, polylysine, spermine, spermidine, protamin, protamin sulfate, oligopeptides, polypeptides, denaturation products of proteins and proteids, such as gelatin, casein hydrolyzates, glutelins; nitrogen-containing polysaccharides such as mucopolysaccharides, glycoproteids, chitins and mixtures thereof, preferably polyethyleneimines (PEI) or polyvinylamines (PVAm).

7. The dispersion according to claim 6, wherein the compounds containing polyamino groups are bound to diagnostically or pharmaceutically effective substances, cell- or tissue-specific substances, cells or cell fusion-mediating substances or gene transfer-mediating substances.

8. The dispersion according to claim 6, wherein the compounds containing polyamino groups are bound to short-lived radiopharmaceutical agents containing ¹¹C, ¹³N, ¹⁵O, ¹⁸F, ⁶⁸Ga, ⁷⁵Br, ¹²³I, preferably [¹¹C]-thymidine, [¹⁸F]-fluoro-L-DOPA, [⁶⁸Ga]-anti-CD66.

9. The dispersion according to any of claim 1, wherein it contains positively charged metal ions selected from positively charged metal ions of the chemical elements copper, silver, gold, iron, gallium, thallium, bismuth, palladium, rhenium, ruthenium, platinum, technetium, indium, iridium, radium, selenium, yttrium, zirconium and rare earths, as well as mixtures thereof, and of the radioactive isotopes ⁵²Fe, ⁶⁷Ga, ^{99m}TC, ¹¹³In, ¹⁸⁸Rh, ¹⁹²Ir, ¹⁹⁸Au, ²⁰¹Tl, ²²³Ra, as well as mixtures thereof.

10. A method for the production of an aqueous dispersion of superparamagnetic single-domain particles of iron hydroxide, iron oxide hydrate, iron oxide, iron mixed oxide or iron with a particle size of from 2 to 10 nm, which particles bear aliphatic di- and/or tricarboxylic acids selected from citric acid, malic acid, tartaric acid, derivatives or mixtures thereof as stabilizer substances on their surface, by precipitation of the superparamagnetic iron-containing particles from aqueous iron salt solutions using an alkali solution or ammonium hydroxide, subsequent treatment with aliphatic di- and/or tricarboxylic acids selected from citric acid, malic acid and tartaric acid, derivatives or mixtures thereof, and purification of the particles thus stabilized using dialysis with distilled water until the dialyzate has an electric conductivity of less than 10 μS/cm, characterized in that the dialyzate is subsequently treated with an aqueous salt solution of aliphatic di- and/or tricarboxylic acids selected from citric acid, malic acid, tartaric acid, derivatives or mixtures thereof and dialyzed with distilled water until the dialyzate has an electric conductivity of less than 10 μS/cm and a content of free iron ions of less than 1 mg/l, subsequently treated with an aqueous solution of the above-mentioned free di- and/or tricarboxylic acids, derivatives or mixtures thereof and dialyzed with distilled water until the dialyzate has an electric conductivity of less than 10 μS/cm and a content of free iron ions of less than 1 mg/l, and N-methyl-D-glucamine (meglumine) and/or 2-amino-2-(hydroxymethyl)-1,3-propanediol (trometamol) are added.

11. The method according to claim 10, wherein citric acid salts and free citric acid are used in the treatment of the dialyzate.

12. The method according to claim 10, wherein the resulting aqueous dispersion is added with a physiologically tolerable iron ions complexing agent, preferably glycerophosphoric acid, ethylenediaminetetraacetic acid (EDTA), N-hydroxyethylethylenediamin-etriacetic acid (HEDTA), diethylenetriaminepentaacetic acid (DTPA), α-mercaptopropionylglycine (thiopronine), 2,3-mercapto-1-propanesulfonic acid, 30-amino-3,14,25-trihydroxy-3,9,14,20,25-pentaazatriacontane-2,10,13,21,24-pentaone-methanesulfonic acid (deferoxamine mesylate) or a mixture or salt thereof, more preferably glycerophosphoric acid or a salt thereof.

13. The method according to claim 10, wherein physiologically tolerable compounds containing polyamino groups, selected from the group of polyethyleneimines (PEI), polyvinylamines (PVAm), PEI and PVAm copolymers, polylysine, spermine, spermidine, protamin, protamin sulfate, oligopeptides, polypeptides, denaturation products of proteins and proteids, such as gelatin, casein hydrolyzates, glutelins; nitrogen-containing polysaccharides such as mucopolysaccharides, glycoproteids, chitins and mixtures thereof, are added, preferably polyethyleneimines (PEI) or polyvinylamines (PVAm).

14. The method according to claim 13, wherein diagnostically or pharmaceutically effective substances, cell- or tissue-specific binding substances, cells or cell fusion-mediating substances or gene transfer-mediating substances are bound to the compounds containing polyamino groups.

15. The method according to claim 13, wherein short-lived radiopharmaceutical agents containing ¹¹C, ¹³N, ¹⁵O, ¹⁸F, ⁶⁸Ga, ⁷⁵Br, ¹²³I, preferably [¹¹C]-thymidine, [¹⁸F]-fluoro-L-DOPA, [⁶⁸Ga]-anti-CD66, are bound to the compounds containing polyamino groups.

16. The method according to claim 10, wherein the resulting aqueous dispersion is added with positively charged metal ions selected from positively charged metal ions of the chemical elements copper, silver, gold, iron, gallium, thallium, bismuth, palladium, rhenium, ruthenium, platinum, technetium, indium, iridium, radium, selenium, yttrium, zirconium and rare earths, as well as mixtures thereof, and of the radioactive isotopes ⁵²Fe, ⁶⁷Ga, ^{99m}Tc, ¹¹³In, ¹⁸⁸Rh, ¹⁹²Ir, ¹⁹⁸Au, ²⁰¹Tl, ²²³Ra, as well as mixtures thereof.

17. A pharmaceutical composition comprising an aqueous dispersion of superparamagnetic single-domain particles of iron hydroxide, iron oxide hydrate, iron oxide, iron mixed oxide or iron in accordance with claim 1.

18. The pharmaceutical composition according to claim 17, wherein it comprises pharmaceutically acceptable adjuvants and/or vehicles.

19. The pharmaceutical composition according to claim 18, wherein the adjuvants and/or vehicles are sugars, preferably mannitol, sorbitol, glucose or xylitol.

20-24. (canceled)

24. Superparamagnetic single-domain particles of iron hydroxide, iron oxide hydrate, iron oxide, iron mixed oxide or iron with a particle size of from 2 to 10 nm, which particles bear aliphatic di- and/or tricarboxylic acids selected from citric acid, malic acid, tartaric acid, derivatives or mixtures thereof as stabilizer substances on their surface and have N-methyl-D-glucamine (meglumine) and/or 2-amino-2-(hydroxymethyl)-1,3-propanediol (trometamol) as cations.
